(19) United States
(12) Patent Application Publication
(10) Pub. No.: US 2013/0090285 A1
(43) Pub. Date: Apr. 11, 2013

(54) METHODS OF TREATMENT WITH GLP-1 RECEPTOR AGONISTS

(71) Applicants: Lynne M. Georgopoulos, Malvern, PA (US); Susan Arnold, Malvern, PA (US)

(72) Inventors: Lynne M. Georgopoulos, Malvern, PA (US); Susan Arnold, Malvern, PA (US)

(73) Assignee: PHASEBIO PHARMACEUTICALS, INC., MALVERM, PA (US)

(21) Appl. No.: 13/534,836

(22) Filed: Jun. 27, 2012

Related U.S. Application Data
(60) Provisional application No. 61/501,499, filed on Jun. 27, 2011.
(60) Provisional application No. 61/596,887, filed on Feb. 9, 2012.
(60) Provisional application No. 61/656,758, filed on Jun. 7, 2012.

(51) Int. Cl. A61K 38/39; A61K 38/26

(52) U.S. Cl. CPC A61K 38/39; A61K 38/26
USPC 514/5.3

(57) ABSTRACT

The invention provides methods for treating diabetes or obesity, as well as methods for inducing weight loss, preventing weight gain, or controlling weight in a patient in need thereof. The methods comprise administering to the patient at least one effective dose of a GLP-1 receptor agonist, or a regimen of GLP-1 receptor agonist comprising a plurality of substantially evenly spaced doses. The effective dose or regimen does not induce substantial nausea or appetite suppression in the patient.

### PRIORITY

This application claims priority to the following U.S. Provisional Applications: No. 61/501,499 filed Jun. 27, 2011, No. 61/596,887 filed Feb. 9, 2012, and No. 61/656,758 filed Jun. 7, 2012, and the contents of each are hereby incorporated by reference in their entirety.

### BACKGROUND

GLP-1 receptor agonists, such as GLP-1 and Exendin-4, have shown promise for treating conditions such as diabetes mellitus and obesity. However, these peptides have a short half-life in the circulation, and when administered at high doses to counter the short half-life, these agents can induce nausea and vomiting. Murphy and Bloom, Nonpeptidic glucagon-like peptide 1 receptor agonists: A magic bullet for diabetes? PNAS Vol. 104(3):689-690 (2007). These adverse effects can interfere with or confound the efficacy of the drug. Thus, alternative GLP-1 receptor agonists and/or formulations thereof are needed to improve treatment.

### SUMMARY OF THE INVENTION

The invention provides methods of treatment with a GLP-1 receptor agonist. In various embodiments, the invention provides a method for treating diabetes or treating or preventing obesity. The method of treatment in various embodiments induces weight loss, prevents weight gain, or helps controls weight in the patient. The methods comprise administering to the patient at least one effective dose of a GLP-1 receptor agonist, or administering a regimen of the effective doses. The effective dose or regimen does not induce substantial nausea or appetite suppression in the patient. In various embodiments, the method does not require or induce a substantial reduction in food intake in the patient to control weight gain, induce weight loss, or prevent weight gain.

In various embodiments of the invention, the GLP-1 receptor agonist is GLP-1 7-36, GLP-1 7-37, exendin, oxyntomodulin, or a derivative or homolog of one or more of the foregoing, including dual GLP-1 receptor/glucagon receptor agonists. The GLP-1 receptor agonist is generally constructed or formulated for sustained release, and/or exhibits slow uptake to the patient's circulation when administered, to thereby avoid induction of nausea and other detrimental side effects. In some embodiments, the GLP-1 receptor agonist is fused or covalently attached to an amino acid sequence that contains structural units that provide the sustained release profile. For example, the amino acid sequence may form hydrogen-bonds through protein backbone groups and/or side chain groups, which together with hydrophobic contributions form a sustained release matrix upon administration. In certain embodiments, the amino acid sequence is an Elastin-Like-Peptide (ELP) sequence. The ELP sequence comprises or consists of structural peptide units or sequences that are related to, or mimics of, the elastin protein, and may exhibit an inverse phase transition upon administration. That is, the amino acid sequence may be structurally disordered and highly soluble in the formulation below a transition temperature (Tt), but exhibit a sharp (2-3° C. range) disorder-to-order phase transition when the temperature of the formulation is raised above the Tt. In certain embodiments, the amino acid sequence comprises 90 or 120 repeat units of VPGXG, where each X is selected from V, G, and A, and where the ratio of V:G:A may be about 5:3:2. Other sustained release fusion partners, formulations, and means for obtaining a sustained release profile are described herein.

The GLP-1 receptor agonist may be administered as a regimen of weekly administrations, or as a regimen of twice weekly administrations, or a regimen of administrations given three times weekly, or a regimen of about daily administrations, to thereby control weight gain or induce weight loss without nausea or appetite suppression. In some embodiments, administrations are about every other week, about monthly, etc. In some embodiments, the GLP-1 receptor agonist is administered by subcutaneous injection, and in a manner that avoids a spike in serum level that could potentially induce nausea in the patient. By administering the GLP-1 receptor agonist regimen described herein, therapeutic serum levels of GLP-1 receptor agonist are substantially maintained for a period of time (e.g., without rapid spikes in serum GLP-1 levels) so as to avoid inducing nausea or other side effects, while also inducing beneficial effects such as controlling blood sugar, preventing weight gain, inducing weight loss, and without substantial appetite suppression.

In another aspect, the invention provides a unit dose of a GLP-1-ELP fusion protein, or a kit comprising a plurality (e.g., at least four) of the unit doses. The ELP is as described herein, and may comprise from 90 to 180 repeat units, such as 120 repeat units. The repeat units may be tandem repeats of VPGXG, where X is V, G, and A at the ratio of about 5:3:2. The unit dose contains 50 mg to 100 mg of the recombinant fusion protein (e.g., 50 mg to 100 mg of the fusion protein of SEQ ID NO: 13). As disclosed herein, a unit dose of from 50 mg to 100 mg allows for once weekly administration, while preventing spikes or rises in serum levels that have nausea-inducing potential.

### DESCRIPTION OF THE FIGURES

FIG. 1 shows prevention of weight gain in diabetic mice with administration of a GLP-1-ELP fusion protein (PB1023, or Glymera™). Panel A shows the weight of mice on a high calorie diet, and compares Glymera™ administration (▪) with saline (♦). Panel B shows that Glymera™ prevented weight gain, despite no changes in the food consumption from saline.

FIG. 2 shows that the effect of Glymera™ (▪) in preventing weight gain (as compared to saline (♦)) is reversible upon decrease or cessation of dosing. The grey vertical line on the left shows the point of decreasing dosing concentration, while the black vertical line on the right shows the point of dosing cessation.

FIG. 3 shows that Glymera™ lowers postprandial glucose (Panel A) and lowers blood glucose in the oral glucose tolerance test (Panel B) in diabetic mice. At each time point, bars on right represent GLP-1 administration while bars on the left are saline.

FIG. 4 shows the subject demographics and disposition in Example 2.

FIG. 5 shows the schedule of key study activities in Example 2.

FIG. 6 shows that Glymera™ displays a slow absorption and long half-life when administered by subcutaneous injection in human patients.

FIG. 7 shows Glymera™ concentration in serum following a first dose (Panel A) and following a 4^{th} dose (Panel B), with once weekly subcutaneous administration. Steady sate is reached after the second dose. FIGS. 7A and 7B show the arithmetic mean serum concentrations of Glymera™ following the first (FIG. 7A) and fourth (FIG. 7B) once weekly subcutaneous dose of 0.3, 0.6, 0.9, 1.35 mg/kg to adult subjects with type 2 diabetes mellitus—semi-logarithmic axes.

FIG. 8 shows the relationship between individual subject AUC (inf) and total dose following the fourth subcutaneous dose of Glymera™.

FIG. 9 shows the average CGM glucose response by Glymera™ dose group with Emax model. The dose and AG data fit an Emax model with Emax of −51 mg/dL, and 80% of that effect (ED80) at a dose of 0.85 mg/kg are displayed.

FIG. 10 shows the mean (SEM) change from baseline in fasting plasma glucose over 7 days following the 4^{th} sc dose of Glymera™ (placebo adjusted). The top curve (broken lines) is placebo, the first curve below placebo is 0.6 mg/kg, the second curve below placebo is 0.9 mg/kg, and the third curve below placebo is 1.35 mg/kg.

FIG. 11 shows the mean (SEM) change from baseline in glucose AUC₀₋₂₄₀ minutes (baseline adjusted at 0 minutes) following liquid meal challenge 24 hours following the 4^{th} subcutaneous dose of Glymera™.

FIG. 12 shows the mean (SEM) change from baseline in average glucose measured by CGMs over 7 days following the 4^{th} sc dose of Glymera™ (placebo adjusted). The top curve (broken lines) is placebo, the first curve below placebo is 0.6 mg/kg, the second curve below placebo is 0.9 mg/kg, and the third curve below placebo is 1.35 mg/kg.

FIG. 13 shows the mean 24 hour CGM curves, post (following 4^{th} dose) and pre comparisons between placebo and 1.35 mg/kg of Glymera™. The top curve is dose 0 (post-pre), the second from the top curve is dose 1.35 (post-pre), the third from the top curve is dose 1.35-dose 0 (post-pre). The horizontal line near −60 is the 95% MADz (one-tailed).

FIG. 14 compares the PK profiles of two subjects that experienced GI (nausea and vomiting) side effects with the average PK profiles. In cohort 3, mild nausea was reported starting ˜6 hours post-dose and reported resolved at around 43 hours post-dose. In cohort 5, mild nausea was reported starting ˜2 hours post-dose (duration ˜4 days) and moderate vomiting at ˜4 hours post-dose duration ˜8 hours).

FIG. 15 shows adverse events reported in ≧2 subjects receiving multiple doses of Glymera™ or ≧2 subjects receiving placebo.

FIG. 16 shows the probability of nausea as a function of PK and rate(pk). Up to 1000 ng/ml (˜ED86), the nausea risk depends very little on absolute concentration, but is sensitive to the speed of increasing plasma drug concentration.

FIG. 17 shows the probability of nausea as a function of PK and rate(pk) over a larger PK range. Within the observed range of absolute concentrations, the nausea risk increases considerably at the doses above those needed for glucose lowering effect.

FIG. 18 shows the probability of nausea as a function of PK and rate(pk) over a still larger PK range. Including extrapolated data in the full model, very high absolute concentration of the GLP-1 fusion makes nausea almost a certainty, overwhelming the speed of increase as a precipitating factor.

### DETAILED DESCRIPTION OF THE INVENTION

In various aspects, the invention provides a method for treating a patient with a GLP-1 receptor agonist. The method induces weight loss, prevents weight gain, and/or controls weight in the patient, which may be in addition to other therapeutic effects of GLP-1 receptor agonist. The methods comprise administering at least one effective dose of a GLP-1 receptor agonist, or a regimen comprising a plurality of doses, such as substantially evenly spaced doses, to the patient. The effective dose or regimen does not induce substantial appetite suppression or other side effects in the patient. In another aspect, the invention provides a method for controlling weight in a patient in need thereof. In this aspect, the method comprises administering a GLP-1 receptor agonist regimen to the patient, where the regimen is effective to maintain therapeutic serum levels of the GLP-1 receptor agonist below a nausea-inducing threshold. The regimen is also effective to avoid a rapid (nausea-inducing) rise in GLP-1 receptor agonist serum levels. Generally, the effective dose or regimen does not require or induce a substantial reduction in food intake in the patient to control weight gain, induce weight loss, or prevent weight gain.

In certain embodiments, the patient is overweight or obese, and is therefore in need of weight loss, prevention of weight gain, and weight control overall. For example, the patient in some embodiments has a body mass index of from 25 to 30, or a body mass index of at least 30. In some embodiments, the patient has a body mass index of at least 35. Body mass index or BMI is a measure of body fat based on height and weight, and the determination of BMI is well known.

In some embodiments, the patient has a positive energy balance prior to treatment, and thus is likely to continue weight gain absent treatment. In these embodiments, the invention may provide for weight loss or weight control without substantial changes in calorie intake. It is believed, without wishing to be bound by theory, that the invention provides, in some embodiments, underlying changes to the patient's metabolism to enable weight loss or weight control.

In embodiments of the invention, the patient has metabolic disease. Metabolic disease maybe defined by the presence in the patient of at least two of:
(1) triglycerides>150 mg/dL (1.7 mmol/L);
(2) HDL cholesterol<40 mg/dL (1.03 mmol/L) for a male, <50 mg/dL (1.29 mmol/L) for a female;
(3) systolic BP>130 or diastolic BP>85 mm Hg, or the patient is being treated for hypertension;
(4) fasting plasma glucose (FPG)>100 mg/dL (5.6 mmol/L) or the patient is being treated for hyperglycemia; and
(5) an elevated waist circumference equal to or greater than 40 inches (102 cm) for men, or equal to or greater than 35 inches (88 cm) for women.

In accordance with embodiments of the invention, the patient's caloric intake is not significantly altered by the treatment. For example, the patient may be placed on (or continue with) a diet that is not restricted in caloric intake by more than 20%, or not by more than 10%, or not by more than 5%, of the patient's caloric intake at the start of treatment. The caloric intake in conjunction with the treatment is sufficient to achieve a neutral or negative energy balance for the patient. That is, in various embodiments, the effective dose or regimen of the GLP-1 receptor agonist affects the patient's metabolic rate to induce weight loss or prevent weight gain without substantial reduction in caloric intake and/or water intake.

In various embodiments, the patient may or may not be diabetic (type 1 or type 2) or may or may not be pre-diabetic. In some embodiments, the patient is diabetic (type 1 or type 2), and the benefits in weight control described herein are secondary to the treatment of diabetes. The effective dose or regimen of the GLP-1 receptor agonist as described herein may have a substantially neutral impact on the first phase insulin response in the patient, and/or may have a substantially enhanced effect on the second phase insulin response in the patient. Beta cells in the islets of Langerhans release insulin in two phases. The first phase release is rapidly triggered in response to increased blood glucose levels. The second phase is a sustained, slow release of newly formed vesicles triggered independently of sugar.

In some embodiments of the invention, the GLP-1 receptor agonist is GLP-1 7-36, GLP-1 7-37, exendin, or a derivative or homolog of one of the foregoing. For example, the GLP-1 receptor agonist may have from 1 to about 5 amino acid insertions, deletions, additions or truncations (collectively) relative to GLP-1 7-36, GLP-1 7-37, or exendin, such as 1, 2, or 3 amino acid insertions, deletions, additions or truncations (collectively). Exemplary derivatives of GLP-1 and Exendin-4 are disclosed in US Patent Publication No. 2010/0022455, which is hereby incorporated by reference. GLP-1 [7-36] has the following amino acid sequence: HAEGTFTSDVSSYLEGQAAKEFIAWLVKGR (SEQ ID NO: 14). The GLP-1 may contain glycine (G) at the second position, giving, for example, the sequence HGEGTFTSDVSSYLEGQAAKEFIAWLVKGR (SEQ ID NO: 15). Exendin-4 has the following amino acid sequence:

(SEQ ID NO: 16)
HGEGTFTSDLSKQMEEEAVRLFEWLKNGGPSSGAPPPS.

In some embodiments, the GLP-1 receptor agonist is a GLP-1 receptor co-agonist, in that the GLP-1 receptor agonist is also an agonist of one or more of the glucagon, GIP, or GLP-2 receptors. For example, the GLP-1 receptor agonist may be a dual GLP-1 receptor/glucagon receptor agonist. For example, the GLP-1 receptor agonist may be oxyntomodulin, which has the amino acid sequence HSQGTFTSDYSKYLDSRRAQDFVQWLMNTKRNKNNIA (SEQ ID NO: 17), optionally having from 1 to about 5 amino acid insertions, deletions, additions or truncations (collectively) relative to SEQ ID NO: 17, such as 1, 2, or 3 amino acid insertions, deletions, additions or truncations (collectively). In some embodiments, the GLP-1 receptor agonist is a dual agonist having an amino acid sequence described in US 2011/0257092, which is hereby incorporated by reference in its entirety. For example, the dual agonist may comprise the amino acid sequence HSEGTFTSDYSKYLDSRRAQDFVQWLMNTKRNRNNIA (SEQ ID NO:18), optionally with from 1 to about 5 amino acid insertions, deletions, additions or truncations (collectively) relative to SEQ ID NO:18, such as 1, 2, or 3 amino acid insertions, deletions, additions or truncations (collectively). Other dual or multi receptor agonists are described in US 2011/016602 and US 2010/00190701, each of which is hereby incorporated by reference, in particular with regard to the structures and sequences of GLP-1 receptor co-agonists described therein. Additional descriptions of GLP-1 receptor co-agonists can be found in Pocai A et al., Glucagon-Like Peptide 1/Glucagon Receptor Dual Agonism Reverses Obesity in Mice, Diabetes 58:2258-2266 (2009) and Patterson J T, et al., Functional association of the N-terminal residues with the central region in glucagon-related peptides, J. Pept. Sci. 17:659-666 (2011), each of which are hereby incorporated by reference in their entirety.

The GLP-1 receptor agonist is generally constructed or formulated for sustained release, and/or exhibits slow uptake to the patient's circulation when administered, to thereby avoid induction of nausea and/or appetite suppression. Various means for sustained release of drugs are known, and may be used in accordance with the invention to provide the PK profile described herein. For example, the sustained release formulation may involve incorporation of the GLP-1 receptor agonist in microspheres and/or complexation with zinc, a transdermal patch, or a formulation described in WO 2007/139589, which is hereby incorporated by reference. Alternatively, the slow uptake is effected by fusion of the GLP-1 receptor agonist to a second protein such as a serum protein, examples of which include a transferrin amino acid sequence, an Ig Fc amino acid sequence (e.g., IgG2 Fc), or an albumin amino acid sequence.

In some embodiments, the GLP-1 receptor agonist is fused or attached (e.g., by recombinant fusion or chemical conjugation) to an amino acid sequence that contains structural units that form hydrogen-bonds through protein backbone groups and/or side chain groups to form a sustained release matrix upon administration. Hydrophobic interactions may also contribute to matrix formation. This strategy for sustained release includes formulations for sustained release that are described in U.S. Provisional Application No. 61/551,506, filed Nov. 14, 2011, which is hereby incorporated by reference in its entirety. In some embodiments of this formulation, the amino acid side chains do not contain hydrogen bond donor groups, with hydrogen bonds being formed substantially through the protein backbone. Exemplary amino acids include proline, alanine, valine, glycine, and isoleucine, and similar amino acids. In some embodiments, the structural units are substantially repeating structural units, so as to create a substantially repeating structural motif, and substantially repeating hydrogen-bonding capability. In these and other embodiments, the amino acid sequence contains at least 10%, at least 20%, at least 40%, or at least 50% proline, which may be positioned in a substantially repeating pattern. In this context, a substantially repeating pattern means that at least 50% or at least 75% of the proline residues of the amino acid sequence are each part of a single definable structural unit. In still other embodiments, the amino acid sequence contains amino acids with hydrogen-bond donor side chains, such as serine, threonine, and/or tyrosine. In some embodiments, the repeating sequence may contain from one to about four proline residues, with remaining residues independently selected from non-polar residues, such as glycine, alanine, leucine, isoleucine, and valine. Non-polar or hydrophobic residues may contribute hydrophobic interactions to the formation of the matrix. The amino acid sequences driving the sustained release may form a “gel-like” state upon injection at a temperature higher than the storage temperature.

In some embodiments, the amino acid sequence capable of forming the matrix at body temperature is a peptide having repeating units of from four to ten amino acids. The repeating unit may form one, two, or three hydrogen bonds in the formation of the matrix. In certain embodiments, the amino acid sequence capable of forming the matrix at body temperature is an amino acid sequence of silk, elastin, collagen, or keratin, or mimic thereof, or an amino acid sequence disclosed in U.S. Pat. No. 6,355,776, which is hereby incorporated by reference.

In certain embodiments, the amino acid sequence is an Elastin-Like-Protein (ELP) sequence. The ELP sequence comprises or consists of structural peptide units or sequences that are related to, or mimics of, the elastin protein. The ELP sequence is constructed from structural units of from three to about twenty amino acids, or in some embodiments, from four to ten amino acids, such as four, five or six amino acids. The length of the individual structural units may vary or may be uniform. Exemplary structural units include units defined by SEQ ID NOs: 1-12 (below), which may be employed as repeating structural units, including tandem-repeating units, or may be employed in some combination. Thus, the ELP may comprise or consist essentially of structural unit(s) selected from SEQ ID NOs: 1-12, as defined below.

In some embodiments, including embodiments in which the structural units are ELP units, the amino acid sequence comprises or consists essentially of from about 10 to about 500 structural units, or in certain embodiments about 50 to about 200 structural units, or in certain embodiments from about 80 to about 200 structural units, or from about 80 to about 150 structural units, such as one or a combination of units defined by SEQ ID NOs: 1-12. Thus, the structural units collectively may have a length of from about 50 to about 2000 amino acid residues, or from about 100 to about 800 amino acid residues, or from about 200 to about 700 amino acid residues, or from about 400 to about 600 amino acid residues.

The amino acids sequence may exhibit a reversible inverse phase transition with the selected formulation. This reversible inverse phase transition may be visible in vitro in some embodiments. That is, the amino acid sequence may be structurally disordered and highly soluble in the formulation below a transition temperature (Tt), but exhibit a sharp (2-3° C. range) disorder-to-order phase transition when the temperature of the formulation is raised above the Tt. In addition to temperature, length of the amino acid polymer, amino acid composition, ionic strength, pH, pressure, selected solvents, presence of organic solutes, and protein concentration may also affect the transition properties, and these may be tailored in the formulation for the desired absorption profile. Exemplary formulations are described in U.S. Provisional Application No. 61/551,506, filed Nov. 14, 2011, and such description is hereby incorporated by reference. The absorption profile can be easily tested by determining plasma concentration or activity of the active agent over time.

In certain embodiments, the ELP component(s) may be formed of structural units, including but not limited to:
(a) the tetrapeptide Val-Pro-Gly-Gly, or VPGG (SEQ ID NO: 1);
(b) the tetrapeptide Ile-Pro-Gly-Gly, or IPGG (SEQ ID NO: 2);
(c) the pentapeptide Val-Pro-Gly-X-Gly (SEQ ID NO: 3), or VPGXG, where X is any natural or non-natural amino acid residue, and where X optionally varies among polymeric or oligomeric repeats;
(d) the pentapeptide Ala-Val-Gly-Val-Pro, or AVGVP (SEQ ID NO: 4);
(e) the pentapeptide Ile-Pro-Gly-X-Gly, or IPGXG (SEQ ID NO: 5), where X is any natural or non-natural amino acid residue, and where X optionally varies among polymeric or oligomeric repeats;
(f) the pentapeptide Ile-Pro-Gly-Val-Gly, or IPGVG (SEQ ID NO: 6);
(g) the pentapeptide Leu-Pro-Gly-X-Gly, or LPGXG (SEQ ID NO: 7), where X is any natural or non-natural amino acid residue, and where X optionally varies among polymeric or oligomeric repeats;
(h) the pentapeptide Leu-Pro-Gly-Val-Gly, or LPGVG (SEQ ID NO: 8);
(i) the hexapeptide Val-Ala-Pro-Gly-Val-Gly, or VAPGVG (SEQ ID NO: 9);
(j) the octapeptide Gly-Val-Gly-Val-Pro-Gly-Val-Gly, or GVGVPGVG (SEQ ID NO: 10);
(k) the nonapeptide Val-Pro-Gly-Phe-Gly-Val-Gly-Ala-Gly, or VPGFGVGAG (SEQ ID NO: 11); and
(l) the nonapeptides Val-Pro-Gly-Val-Gly-Val-Pro-Gly-Gly, or VPGVGVPGG (SEQ ID NO: 12).

Such structural units defined by SEQ ID NOs: 1-12 may form structural repeat units, or may be used in combination to form an ELP. In some embodiments, the ELP component is formed entirely (or almost entirely) of one or a combination of (e.g., 2, 3 or 4) structural units selected from SEQ ID NOs: 1-12. In other embodiments, at least 75%, or at least 80%, or at least 90% of the ELP component is formed from one or a combination of structural units selected from SEQ ID NOs: 1-12, and which may be present as repeating units.

In certain embodiments, the ELP contains repeat units, including tandem repeating units, of Val-Pro-Gly-X-Gly (SEQ ID NO: 3), where X is as defined above, and where the percentage of Val-Pro-Gly-X-Gly (SEQ ID NO: 3) units taken with respect to the entire ELP component (which may comprise structural units other than VPGXG (SEQ ID NO: 3)) is greater than about 50%, or greater than about 75%, or greater than about 85%, or greater than about 95% of the ELP. The ELP may contain motifs of 5 to 15 structural units (e.g. about 10 structural units) of SEQ ID NO: 3, with the guest residue X varying among at least 2 or at least 3 of the units in the motif. The guest residues may be independently selected, such as from non-polar or hydrophobic residues, such as the amino acids V, I, L, A, G, and W (and may be selected so as to retain a desired inverse phase transition property).

In some embodiments, the ELP may form a β-turn structure. Exemplary peptide sequences suitable for creating a β-turn structure are described in International Patent Application PCT/US96/05186, which is hereby incorporated by reference in its entirety. For example, the fourth residue (X) in the sequence VPGXG (SEQ ID NO: 3), can be altered without eliminating the formation of a β-turn.

The structure of exemplary ELPs may be described using the notation ELPk [XiYj-n], where k designates a particular ELP repeat unit, the bracketed capital letters are single letter amino acid codes and their corresponding subscripts designate the relative ratio of each guest residue X in the structural units (where applicable), and n describes the total length of the ELP in number of the structural repeats. For example, ELP1 [V5A2G3-10] designates an ELP component containing 10 repeating units of the pentapeptide VPGXG (SEQ ID NO: 3), where X is valine, alanine, and glycine at a relative ratio of about 5:2:3; ELP1 [K1V2F1-4] designates an ELP component containing 4 repeating units of the pentapeptide VPGXG (SEQ ID NO: 3), where X is lysine, valine, and phenylalanine at a relative ratio of about 1:2:1; ELP1 [K1V7F1-9] designates a polypeptide containing 9 repeating units of the pentapeptide VPGXG (SEQ ID NO: 3), where X is lysine, valine, and phenylalanine at a relative ratio of about 1:7:1; ELP1 [V-5] designates a polypeptide containing 5 repeating units of the pentapeptide VPGXG (SEQ ID NO: 3), where X is valine; ELP1 [V-20] designates a polypeptide containing 20 repeating units of the pentapeptide VPGXG (SEQ ID NO: 3), where X is valine; ELP2 [5] designates a polypeptide containing 5 repeating units of the pentapeptide AVGVP (SEQ ID NO: 4); ELP3 [V-5] designates a polypeptide containing 5 repeating units of the pentapeptide IPGXG (SEQ ID NO: 5), where X is valine; ELP4 [V-5] designates a polypeptide containing 5 repeating units of the pentapeptide LPGXG (SEQ ID NO: 7), where X is valine.

With respect to ELP, the Tt is a function of the hydrophobicity of the guest residue. Thus, by varying the identity of the guest residue(s) and their mole fraction(s), ELPs can be synthesized that exhibit an inverse transition over a broad range. Thus, the Tt at a given ELP length may be decreased by incorporating a larger fraction of hydrophobic guest residues in the ELP sequence. Examples of suitable hydrophobic guest residues include valine, leucine, isoleucine, phenylalanine, tryptophan and methionine. Tyrosine, which is moderately hydrophobic, may also be used. Conversely, the Tt may be increased by incorporating residues, such as those selected from: glutamic acid, cysteine, lysine, aspartate, alanine, asparagine, serine, threonine, glycine, arginine, and glutamine.

For polypeptides having a molecular weight >100,000, the hydrophobicity scale disclosed in PCT/US96/05186 (which is hereby incorporated by reference in its entirety) provides one means for predicting the approximate Tt of a specific ELP sequence. For polypeptides having a molecular weight <100,000, the Tt may be predicted or determined by the following quadratic function: Tt=M0+M1X+M2X2 where X is the MW of the fusion protein, and M0=116.21; M1=−1.7499; M2=0.010349.

The ELP in some embodiments is selected or designed to provide a Tt ranging from about 10 to about 37° C. at formulation conditions, such as from about 20 to about 37° C., or from about 25 to about 37° C. In some embodiments, the transition temperature at physiological conditions (e.g., 0.9% saline) is from about 34 to 36° C., to take into account a slightly lower peripheral temperature.

In certain embodiments, the amino acid sequence capable of forming the hydrogen-bonded matrix at body temperature comprises [VPGXG]₉₀, where each X is selected from V, G, and A, and wherein the ratio of V:G:A may be about 5:3:2. For example, the amino acid sequence capable of forming the hydrogen-bonded matrix at body temperature may comprise [VPGXG]₁₂₀, where each X is selected from V, G, and A, and wherein the ratio of V:G:A may be about 5:3:2. 120 structural units of this ELP can provide a transition temperature of just under 37° C. with about 5 to 15 mg/ml (e.g., about 10 mg/ml) of protein (and formulated to be about isotonic with normal saline). At concentrations of about 50 to about 100 mg/mL the phase transition temperature is about 35.5° C. (just below body temperature), which allows for peripheral body temperature to be just less than 37° C.

Alternatively, the amino acid sequence capable of forming the matrix at body temperature comprises [VPGVG]₉₀, or [VPGVG]₁₂₀. 120 structural units of this ELP can provide a transition temperature at about 37° C. with about 0.005 to about 0.05 mg/ml (e.g., about 0.01 mg/ml) of protein.

Elastin-like-peptide (ELP) protein polymers and recombinant fusion proteins can be prepared as described in US 2010/0022455, which is hereby incorporated by reference.

In other embodiments, the amino acid sequence capable of forming the matrix at body temperature may include a random coil or non-globular extended structure. For example, the amino acid sequence capable of forming the matrix at body temperature may comprise an amino acid sequence disclosed in US Patent Publication No. 2008/0286808, WO 2008/155134, US Patent Publication No. 2011/0123487, each of which is hereby incorporated by reference.

For example, in some embodiments the amino acid sequence comprises an unstructured recombinant polymer of at least 40 amino acids. For example, the unstructured polymer may be defined where the sum of glycine (G), aspartate (D), alanine (A), serine (S), threonine (T), glutamate (E) and proline (P) residues contained in the unstructured polymer, constitutes more than about 80% of the total amino acids. In some embodiments, at least 50% of the amino acids are devoid of secondary structure as determined by Chou-Fasman algorithm. The unstructured polymer may comprise more than about 100, 150, 200 or more contiguous amino acids. In some embodiments, the amino acid sequence forms a random coil domain. In particular, a polypeptide or amino acid polymer having or forming “random coil conformation” substantially lacks a defined secondary and tertiary structure.

In accordance with each aspect of the invention, the GLP-1 receptor agonist may be administered at a frequency of from 1 to about 20 times per month, but in various embodiments is administered about weekly, and with a sustained release formulation or delivery means, such as those described herein or incorporated herein by reference. In some embodiments, the GLP-1 receptor agonist is administered two or three times per week, or about daily. In some embodiments, the GLP-1 receptor agonist is administered about every other week or about monthly. While administrations routes can be chosen in accordance with the selected sustained release construct or formulation, in some embodiments the GLP-1 receptor agonist construct or formulation is administered by subcutaneous injection, and in a manner that avoids a spike in serum level that induces nausea (and appetite suppression) in the patient. For example, the GLP-1 receptor agonist containing a fusion of [VPGXG]₁₂₀, where each X is selected from V, G, and A at a ratio of V:G:A of about 5:3:2, formulated at about 5 to 15 mg/ml of protein, and formulated to be about isotonic with normal saline, is administered by subcutaneous injection about weekly for a plurality of months.

By administering the GLP-1 regimen described herein, therapeutic serum levels of GLP-1 receptor agonist are substantially maintained for at least 1 week, or at least 2 weeks, or at least 3 weeks, or for one or a plurality of months without rapid spikes in GLP-1 receptor agonist levels, to thereby avoid inducing nausea and appetite suppression. The therapeutic serum levels of GLP-1 receptor agonist may be substantially maintained for at least 1 month, or at least about 6 months, without inducing nausea.

In exemplary embodiments, the GLP-1 receptor agonist has the amino acid sequence of SEQ ID NO: 13 or is a functional equivalent thereof (e.g., having a transition temperature in the range of 32 to 36° C.), and the dose of the GLP-1 receptor agonist is the dose equivalent of from about 0.1 to about 2.0 mg/kg of SEQ ID NO: 13. For example, in some embodiments, the dose is the dose equivalent of from about 0.3 to about 1.75 mg/kg, or about 0.3 to about 1.5 mg/kg, or about 0.3 to about 1.25 mg/kg of SEQ ID NO: 13. In some embodiments, the dose is the dose equivalent of from about 0.5 to about 2.0 mg/kg, or about 0.5 to about 1.75 mg/kg, or about 0.5 to about 1.5 mg/kg, or about 0.5 to about 1.25 mg/kg of SEQ ID NO: 13. As used herein, the term “dose equivalent” includes a dose of a GLP-1 receptor agonist that is equivalent to the GLP-1 receptor agonist of SEQ ID NO: 13, taking into account differences in mass and relative activity at the GLP-1 receptor. In certain embodiments, the GLP-1 receptor agonist is the GLP-1 receptor agonist of SEQ ID NO: 13, and the GLP-1 receptor agonist is administered at from 50 mg to about 100 mg about once weekly.

As described herein, the GLP-1 regimen provides effective doses and regimens of GLP-1 that do not induce nausea or other side effects in the patient. For example, the serum level that is likely to induce nausea is the equivalent of about 1000 ng/ml of SEQ ID NO: 13. The therapeutic serum level is generally the equivalent of at least about 100 ng/ml of SEQ ID NO: 13. In some embodiments, the therapeutic serum level is the equivalent of at least about 150 ng/ml of SEQ ID NO: 13, or at least about 200 ng/ml of SEQ ID NO: 13, or at least about 300 ng/ml of SEQ ID NO: 13, or at least about 400 ng/ml of SEQ ID NO: 13. The serum level of the GLP-1 receptor agonist may be maintained at a serum level that is the equivalent of from about 100 to about 900 ng/ml of SEQ ID NO: 13 for at least 1 week. In some embodiments, the serum level of the GLP-1 receptor agonist is maintained at a serum level that is the equivalent of from about 100 to about 900 ng/ml of SEQ ID NO: 13 for at least 2 weeks, or at least three weeks, or at least one month, or at least 2, 4, or 6 months.

The various aspects of the invention provide for a sustained release of a GLP-1 receptor agonist. The slow absorption profile provides for a flat PK profile. For example, in various embodiments, the plasma concentration of the active agent over the course of days (e.g., from 2 to about 60 days, or from about 4 to about 30 days) does not change by more than a factor of 10, or by more than a factor of about 5, or by more than a factor of about 3. Generally, this flat PK profile is seen over a plurality of (substantially evenly spaced) administrations, such as at least 2, at least about 5, or at least about 10 administrations of the GLP-1 receptor agonist. In some embodiments, the slow absorption is exhibited by a Tmax (time to maximum plasma concentration) of greater than about 5 hours, greater than about 10 hours, greater than about 20 hours, greater than about 30 hours, or greater than about 50 hours, so as to avoid nausea in the patient.

In some embodiments, the method further comprises administering other agents, such as a glucagon receptor agonist, GLP-2 receptor agonist, and/or GIP receptor agonist to the patient. The glucagon receptor agonist, GLP-2 receptor agonist, and/or GIP receptor agonist may be administered separately from the GLP-1 receptor agonist, or may be co-administered, for example, as part of a mixture or co-formulation. In such embodiments, glucagon receptor agonist, GLP-2 receptor agonist, and/or GIP receptor agonist may also comprise a matrix forming fusion component as described herein, such as an ELP fusion, including that exemplified by SEQ ID NO:13. The glucagon receptor agonist may be oxyntomodulin or analog thereof, glucagon or analog thereof, GIP or analog thereof, or GLP-2 or analog thereof. Such analogs may contain from 1 to about 5 amino acid insertions, deletions, additions or truncations (collectively) relative to the natural sequence, such as 1, 2, or 3 amino acid insertions, deletions, additions or truncations (collectively). Glucagon has the amino acid sequence HSQGTFTSDYSKYLDSRRAQDFVQWLMNT (SEQ ID NO:19). GLP-2 has the amino acid sequence HADGSFSDEMNTILDNLAARDFINWLIQTKITD (SEQ ID NO:20). The amino acid sequence of GIP is YAEGTFISDYSIAMDKIRQQDFVNWLLAQ (SEQ ID NO:21).

In some embodiments, the patient's circulating levels of the GLP-1 receptor agonist are monitored and/or tested at least once to determine the optimal dose and/or dosing schedule to provide the effective dose or regimen. In some embodiments, the induction of nausea and/or appetite suppression in the patient is monitored on the day of dosing one or more times during the treatment period, and the dose or regimen adjusted accordingly.

As exemplary embodiments of the invention, the invention is a method for treating diabetes type 1 or 2, and/or obesity, in a patient in need thereof. The method comprises administering a regimen of a GLP-1 receptor agonist exhibiting and formulated for sustained release and slow uptake to the patient's circulation when administered. The GLP-1 receptor agonist may be as described previously, and can be fused to an elastin-like peptide (ELP) of at least 60, 90 or 120 repeat units, such as that exemplified by SEQ ID NO: 13, which has a transition temperature of just less than 37° C. in normal saline. The GLP-1 receptor agonist may be administered subcutaneously at a frequency of one or two times per week, with this regimen maintained for at least 1 month or at least 6 months to avoid inducing nausea. The dose of the GLP-1 receptor agonist is the dose equivalent of from about 0.1 to about 2.0 mg/kg of SEQ ID NO: 13. For example, where the GLP-1 receptor agonist has the amino acid sequence of SEQ ID NO: 13, the GLP-1 receptor agonist may be administered (e.g., subcutaneously) at about 50 mg to about 100 mg once weekly. Other GLP-1-ELP fusions described herein may be administered at the dose equivalent of 50 mg to 100 mg of SEQ ID NO: 13.

In another aspect, the invention provides a unit dose of a GLP-1-ELP fusion protein, or a kit comprising a plurality (e.g., at least four or at least eight or at least twenty) of the unit doses. The ELP is as described herein, and may comprise from 90 to 180 repeat units, such as 120 repeat units. The repeat units may be tandem repeats of VPGXG, where X is V, G, and A at the ratio of about 5:3:2. The unit dose contains 50 mg to 100 mg of the recombinant fusion protein. For example, the unit dose may have from 50 mg to 100 mg of the fusion protein of SEQ ID NO: 13, or a GLP-1-ELP fusion protein having a similar molecular weight to SEQ ID NO: 13 (e.g., within 20% or within 10% of the molecular weight of SEQ ID NO: 13) and a comparable transition temperature within the range of 34 to 36° C. As disclosed herein, a unit dose of from 50 mg to 100 mg allows for once weekly administration, while preventing spikes or rises in serum levels that have nausea-inducing potential. The unit dose in some embodiments is formulated for subcutaneous administration, such as Normal saline, and may be in the form of pre-dosed pens or the like.

This invention is further illustrated by the following examples that should not be construed as limiting. The contents of all references, patents, and published patent applications cited throughout this application, as well as the Figures and the Sequence Listings, are incorporated herein by reference for all purposes.

### EXAMPLE S

### Example 1

### Diabetic Mouse Model

Glymera™ was administered at 5 mg/kg subcutaneously to diabetic mice (male, 14-15 weeks at start of study). Mice were fed a high calorie diet. Dosing was three times per week.

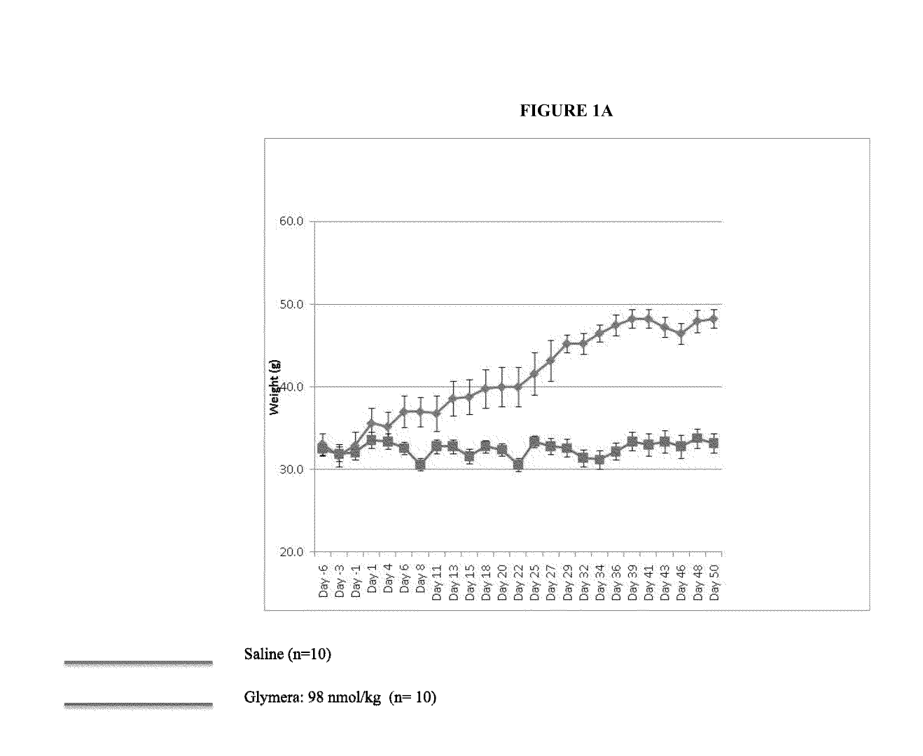

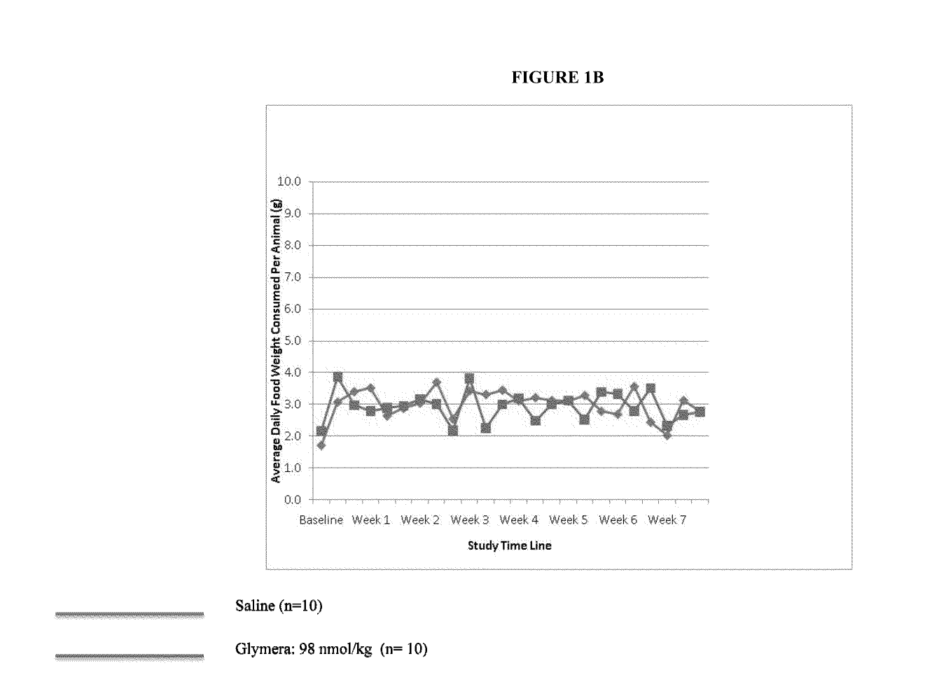

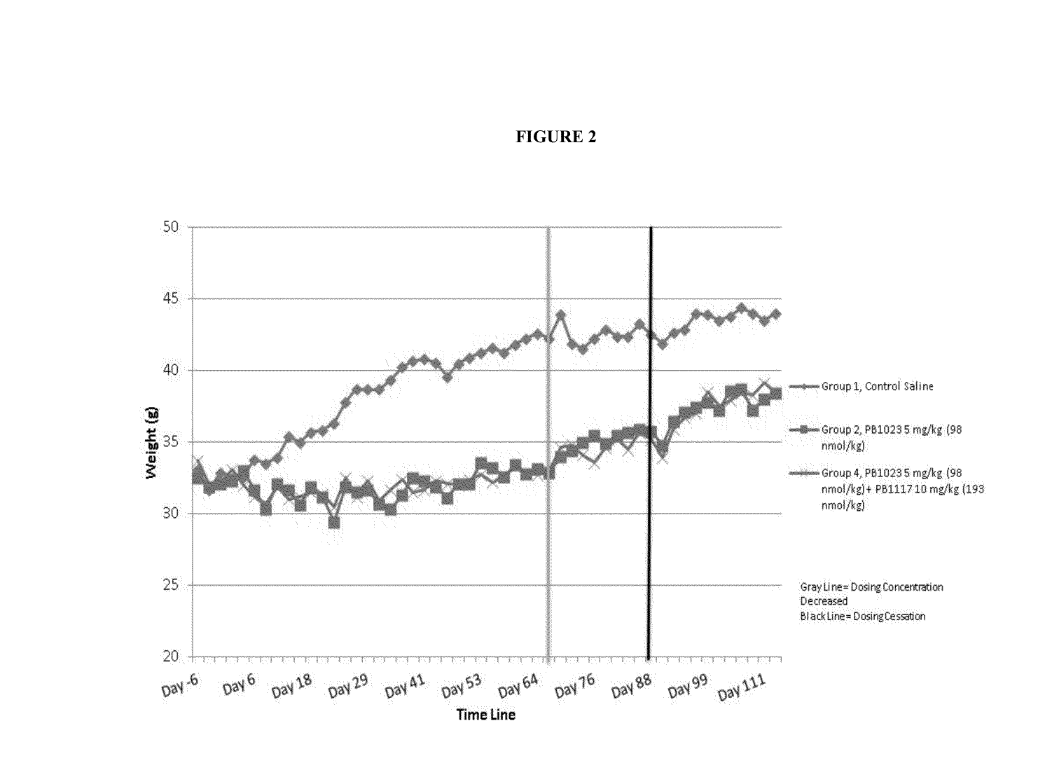

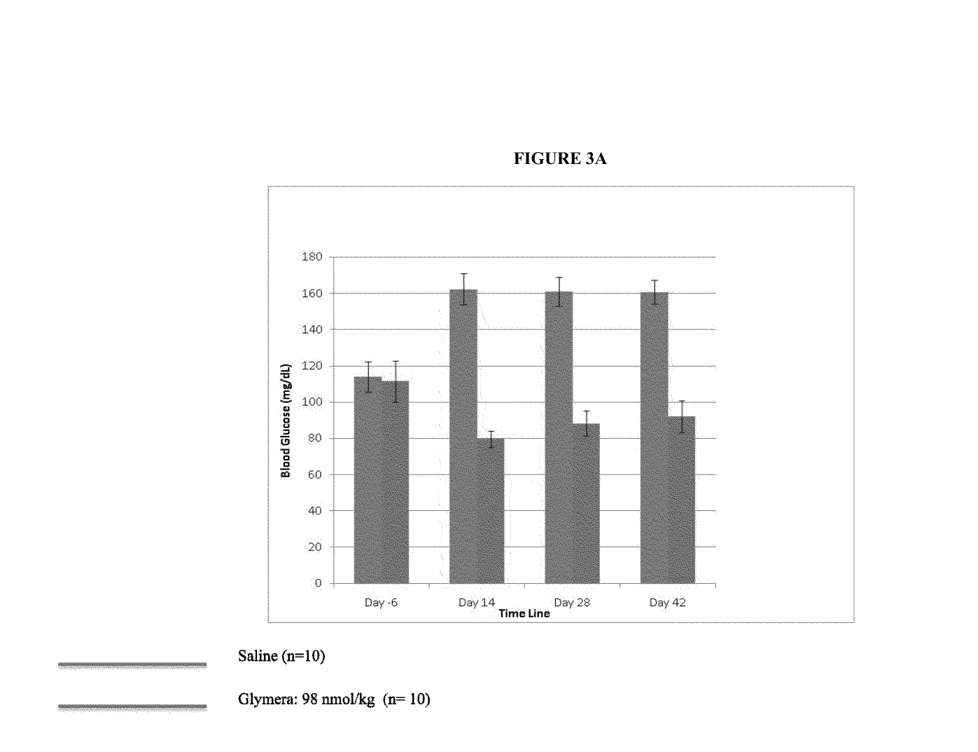

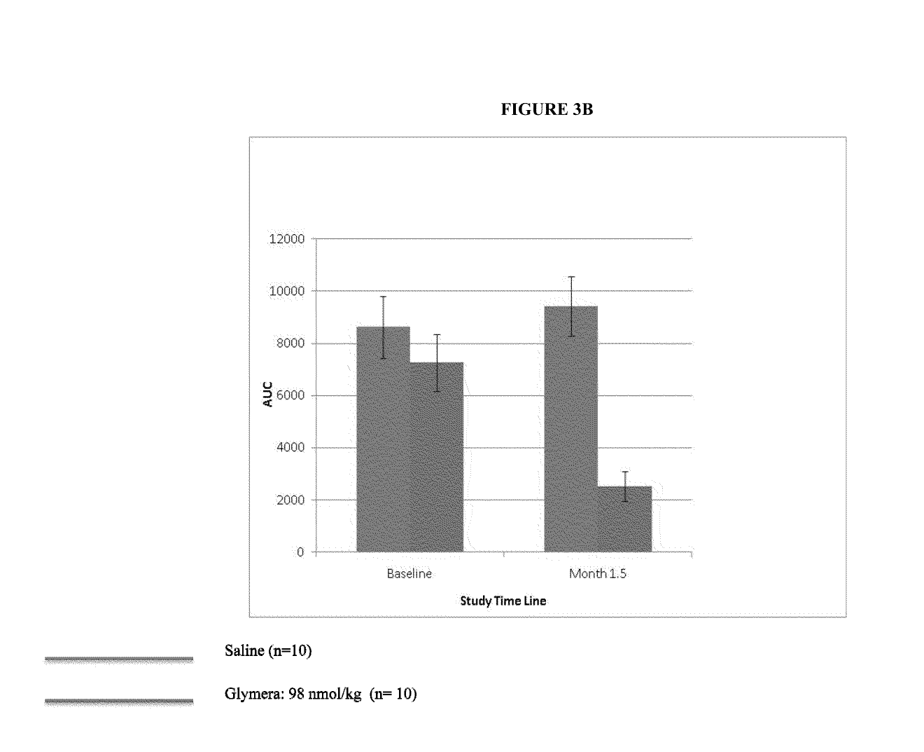

FIG. 1 shows prevention of weight gain in diabetic mice with administration of a GLP-1-ELP fusion protein (PB1023, or Glymera™). Panel A shows the weight of mice on a high calorie diet, and compares Glymera™ administration with saline. Glymera™ prevented weight gain, despite no changes in food consumption from saline (Panel B). The effect of Glymera™ in preventing weight gain is reversible upon cessation of dosing, as shown in FIG. 2. Glymera™ lowers postprandial glucose (FIG. 3A) and lowers blood glucose in the oral glucose tolerance test (FIG. 3B).

### Example 2

### Phase I/II Single Ascending Dose Study and Multiple Ascending Dose Study

In summary, this Phase I/IIa study assessed multiple dose safety, tolerability, pharmacokinetics (PK) and pharmacodynamics (PD) in adults with type II diabetes. Subjects treated with 1 or 2 oral anti-diabetic drugs (OAD) discontinued their OADs during a 2 week run-in period. 56 subjects were randomized to weekly double blind injections of either placebo or Glymera™ for 4 weeks. Subjects were dosed after a liquid mixed meal tolerance test (MMTT). Safety, PK and PD were reviewed before escalation to the next dose of 0.3, 0.6, 0.9, and 1.35 mg/kg, respectively. PK exhibited slow absorption with sustained duration of exposure and minimal accumulation. Dose-response was evident for fasting plasma glucose (FPG), MMTT AUC glucose and average glucose (AG) assessed by continuous glucose monitoring (CGM). At the 1.35 mg/kg dose, placebo-adjusted AG change from baseline was −50 mg/dL (≈−1.8% A1C) (p<0.0001). AG showed minimal loss of efficacy 7 days after the prior dose. CL/F indicated no correlation of clearance to body weight supporting transition to fixed-instead of weight-based dosing. The dose and AG data fit an Emax model with Emax of −48 mg/dL compared to placebo, and 80% of that effect (ED80) at a dose of 63 mg. Glymera™ was well tolerated. The only dose related trend in adverse events (AE) was nausea at the highest doses. 3 subjects experienced mild or moderate injection site erythema that resolved spontaneously. 1 of these received subsequent doses that did not result in exacerbation or recurrent erythema. This subject and 1 other developed low titer non-neutralizing antibodies. There was no indication of adverse effects on any other safety parameters and no serious AEs (SAEs) reported. Therefore, Glymera™ has properties that support development of a once weekly dose.

The Phase 1/2a study (NCT 01236404) was a multicenter randomized, double-blind, placebo-controlled study that was conducted in two parts; Part A as a single ascending dose (SAD) study and Part B as a 4-week multiple (once weekly dosing) ascending dose (MAD) study (topic of this presentation). In the Single Ascending Dose (SAD) (18 active, 6 placebo) study, five dose levels of Glymera™ were evaluated. In the Multiple Ascending Dose (MAD) study, once weekly dosing for four weeks of Glymera™ was evaluated.

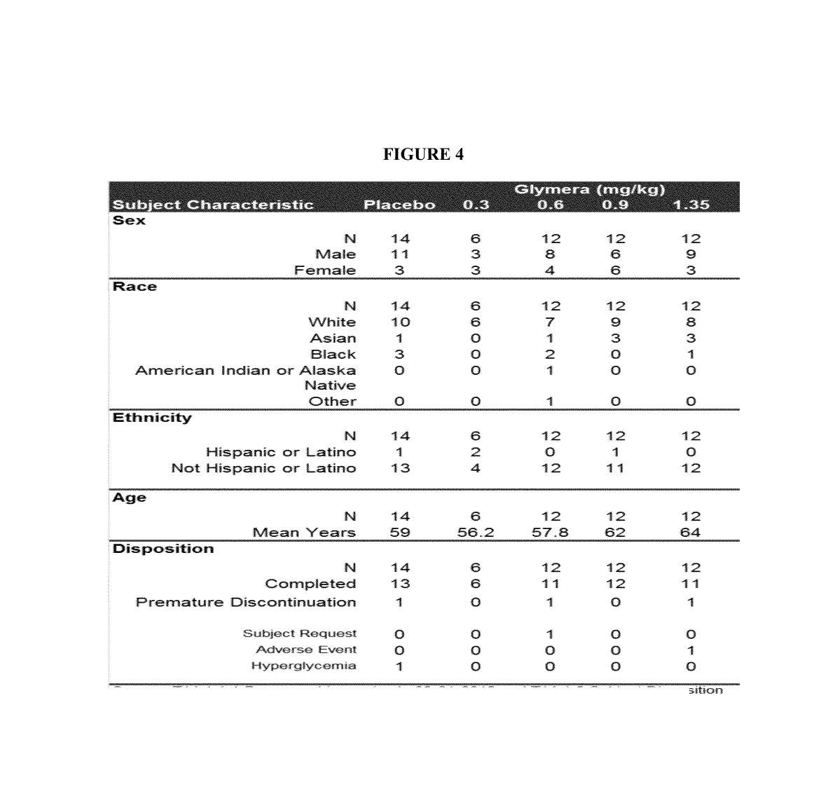

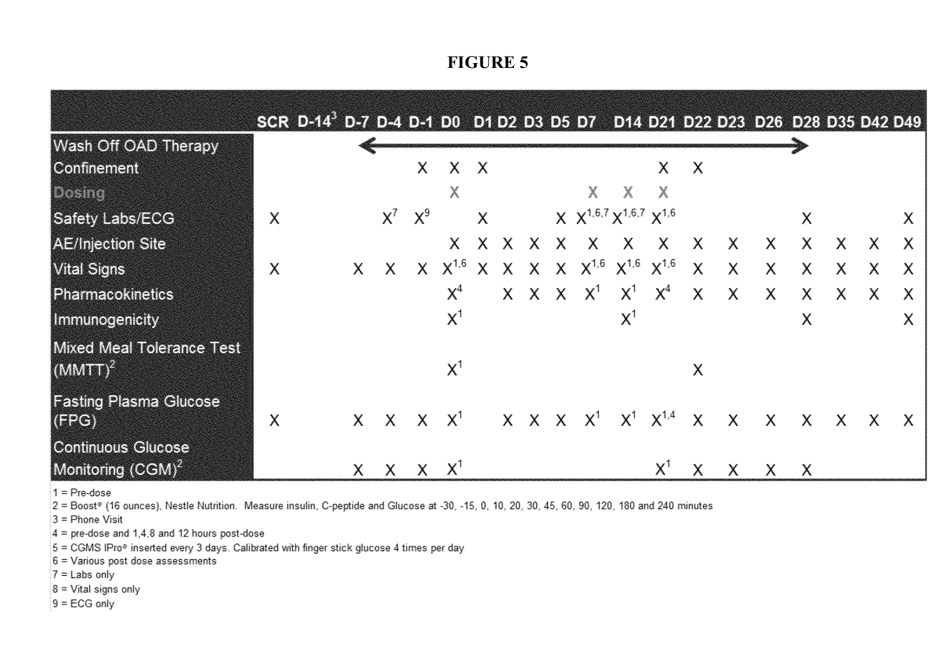

The subjects enrolled were males and females 18-75 years of age with Type 2 Diabetes Mellitus requiring treatment with oral antidiabetic agents (OAD) who were in otherwise stable health (FIG. 4). Subjects on a background of one OAD were required to have a screening HbA1c between 6-9% and between 6-8.5% when taking up to two oral agents. All subjects were required to have a fasting C-peptide of ≧0.8 ng/mL and Body Mass Index (BMI) ≦40 kg/m². Subjects were washed-off from background therapy for a minimum of 14 days prior to dosing with study drug and remained off therapy for 7 days following dosing with study drug (FIG. 5). The purpose of the study was to assess safety, and tolerability as well as to assess the pharmacokinetic and pharmacodynamic profile of various subcutaneous (SC) doses of Glymera™. Subjects participating in the MAD portion of the study underwent assessment of daily fasting glucose monitoring, liquid meal challenge (pre- and ˜24 hours following the 4^{th} dose), and continuous glucose monitoring (CGMS® iPro™, Medtronic, Inc.) for 7 days prior to the first dose and following the 4^{th} dose. Key study activities are described below. A centralized laboratory was used for all analysis of glucose data.

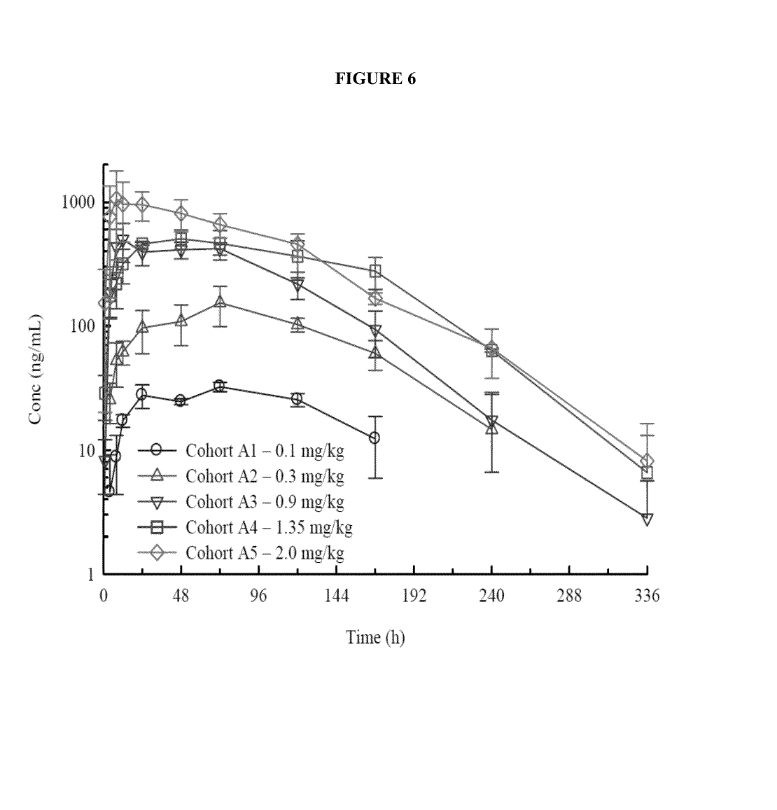

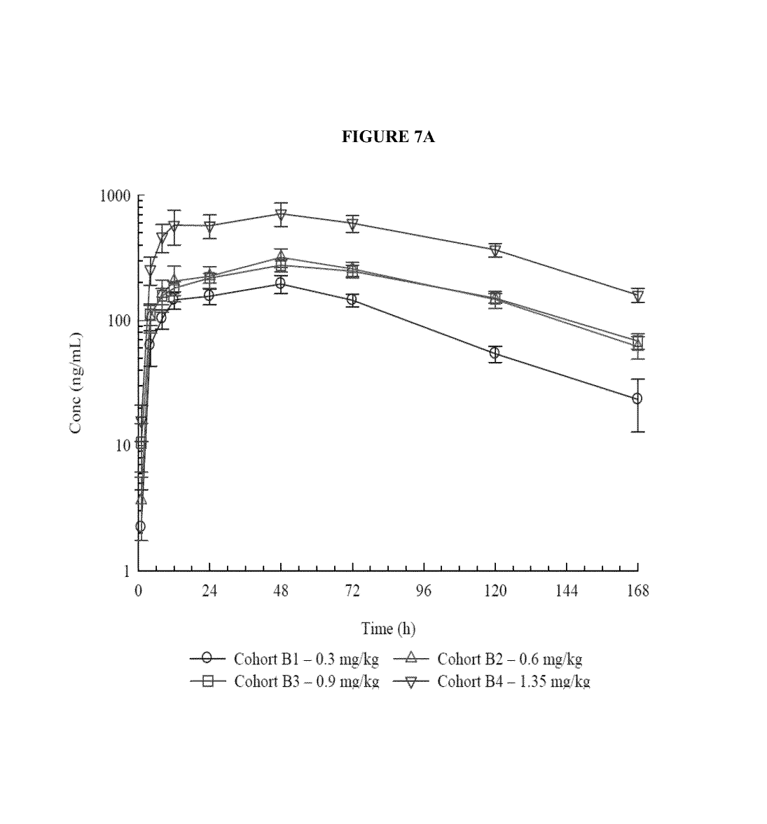

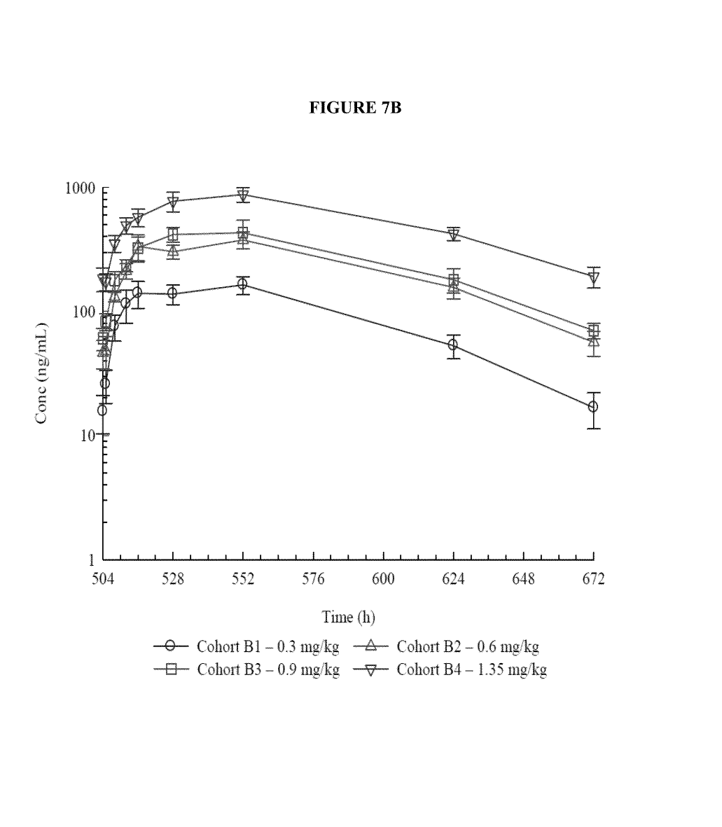

The pharmacokinetic (PK) analysis population consisted of all subjects dosed and who had sufficient data for PK analysis. All subjects were dosed in the abdomen. Depending on the dose level, subjects received between 1 and 9 injections in close proximity in order to deliver a complete dose. Calculations were based on non-compartmental analysis. Serum concentrations less than the validated lower limit of the bioanalytical method were taken as zero for calculation of descriptive statistics at all sampling time-points. Glymera™ displays a slow smooth absorption profile with long half-life that supports weekly subcutaneous dosing. See FIG. 6. Glymera™ concentration in serum following a first dose and following a 4^{th} dose, with once weekly subcutaneous administration, is shown in FIG. 7A, B, respectively. PK showed a combination of a slow absorption and long half-life [flip-flop kinetics] (FIGS. 7A and 7B) following once weekly subcutaneous dosing with Glymera™ Steady state concentration is reached after the second dose with minimal accumulation (˜5%) with repeated administration.

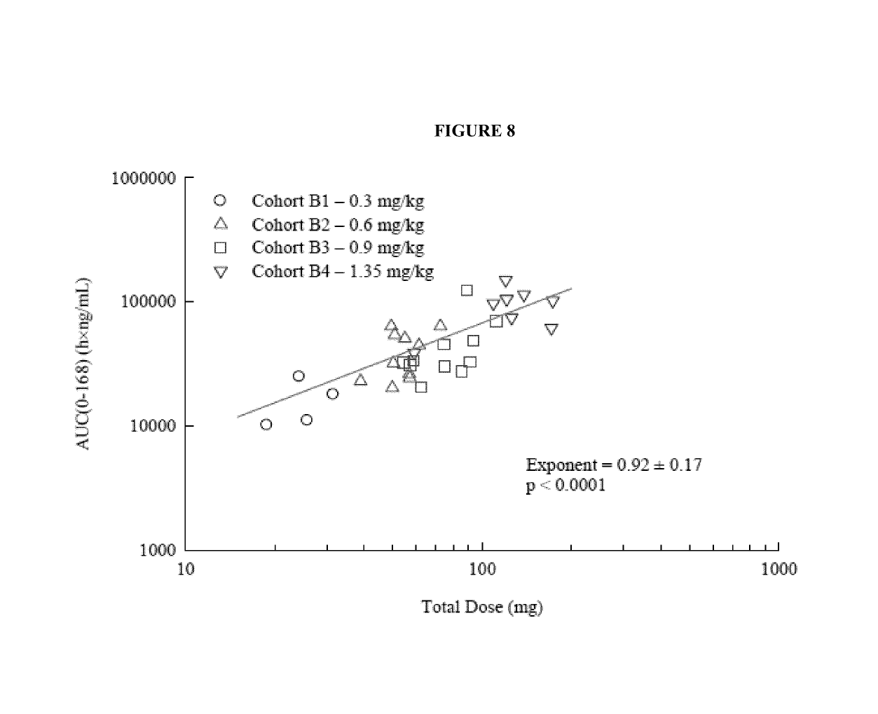

Area under the curve (AUC) showed dose-proportionality (FIG. 8) when plotted versus total dose administered was calculated. There are no apparent relationships between clearance (CL/F), body weight and BMI (data not shown). Based on these data, dosing of Glymera™ is amenable to a once weekly fixed dose regimen.

Glymera™ demonstrated highly statistically significant efficacy as measured by daily fasting glucose and imputed A1c (based on 7 days of continuous glucose monitoring) following 4 weeks of dosing. Glymera™ exhibits a neutral impact on first phase insulin response and an enhanced effect on second phase insulin response.

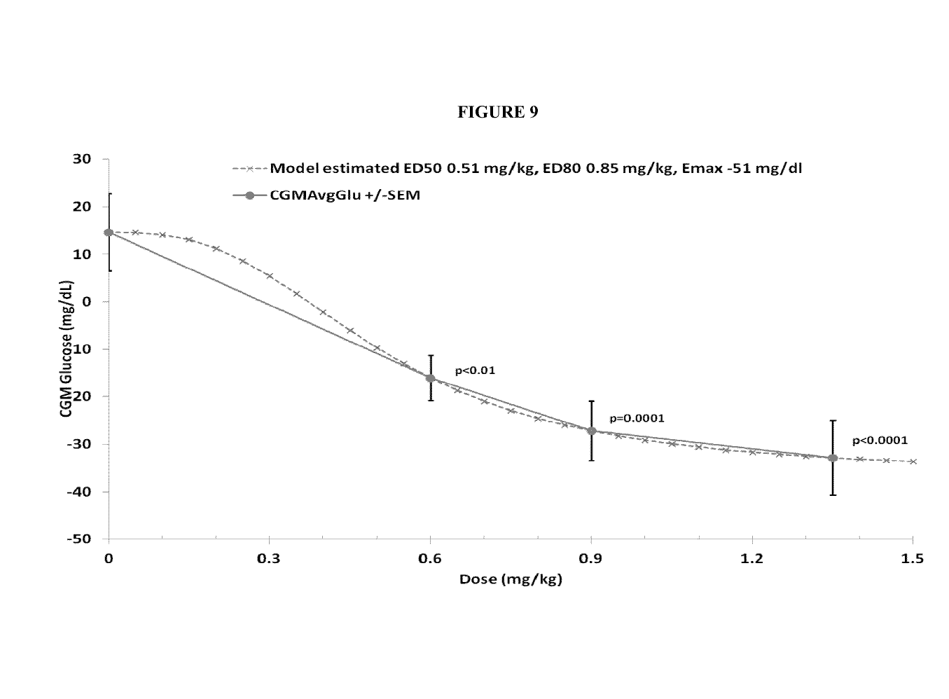

Based on Emax modeling, exposures achieved at the 0.6 mg/kg and 0.9 mg/kg doses would equate to a fixed dose regimen between 50 mg (1000 nmol) and 100 mg (2000 nmol) once weekly. FIG. 9 shows the average CGM glucose response by Glymera™ dose group with Emax model. The dose and AG data fit an Emax model with an Emax of −51 mg/dL, and 80% of that effect (ED80) at a dose of 0.85 mg/kg are displayed.

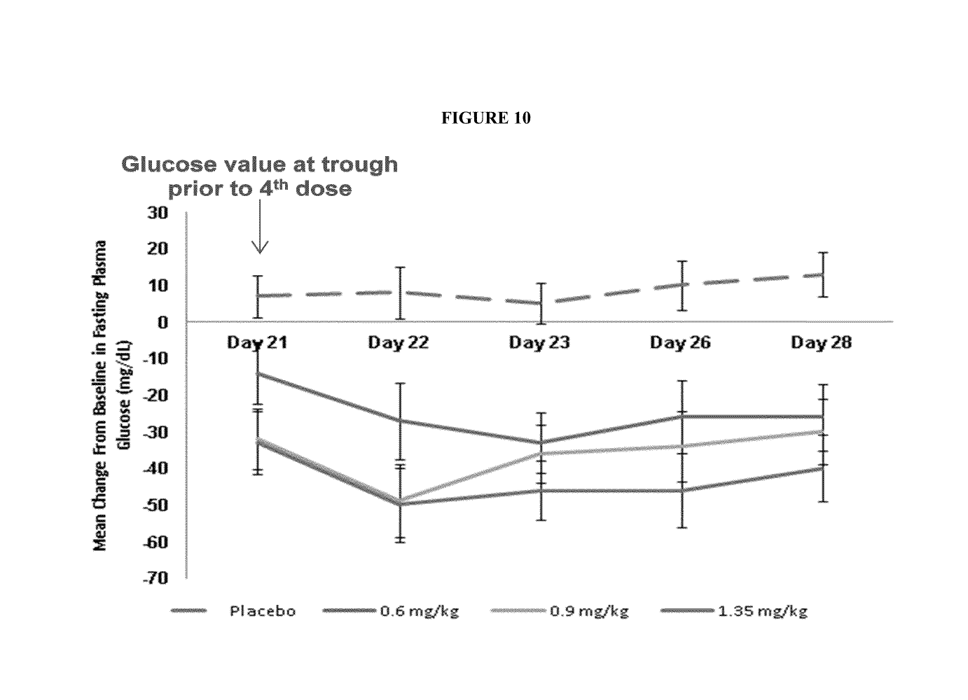

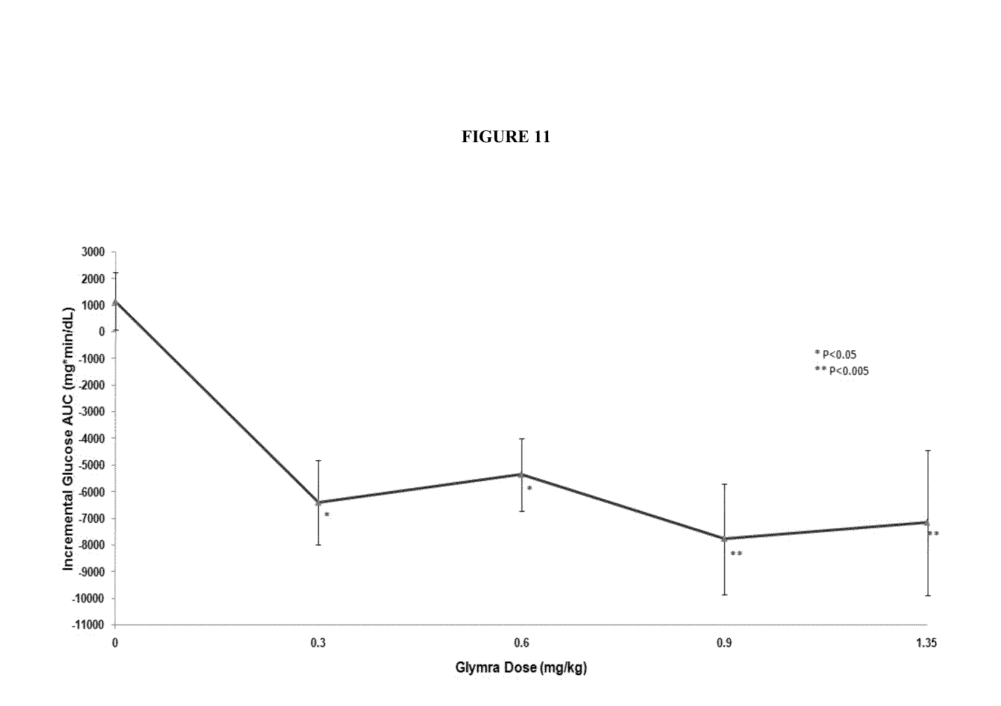

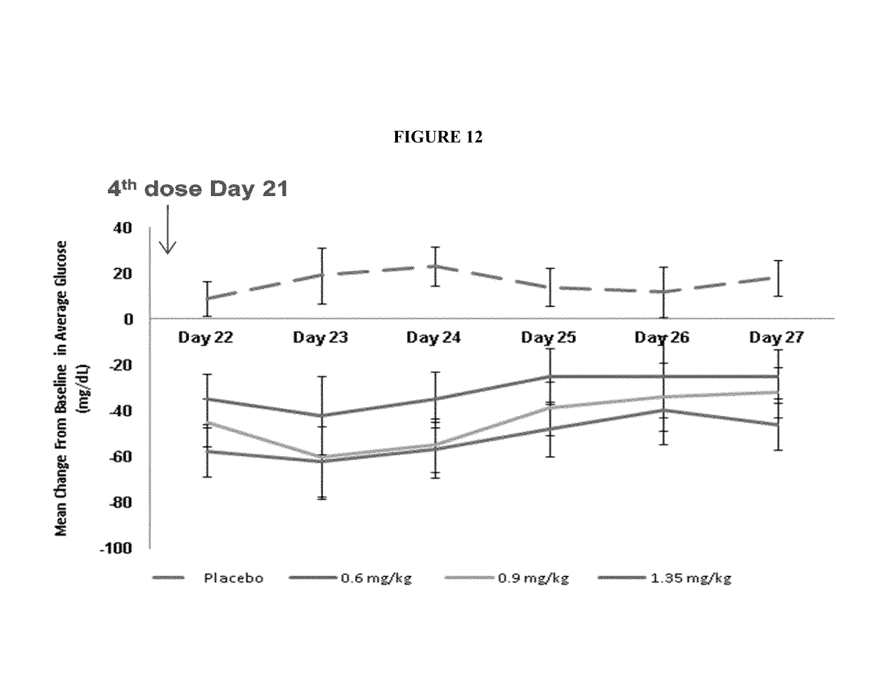

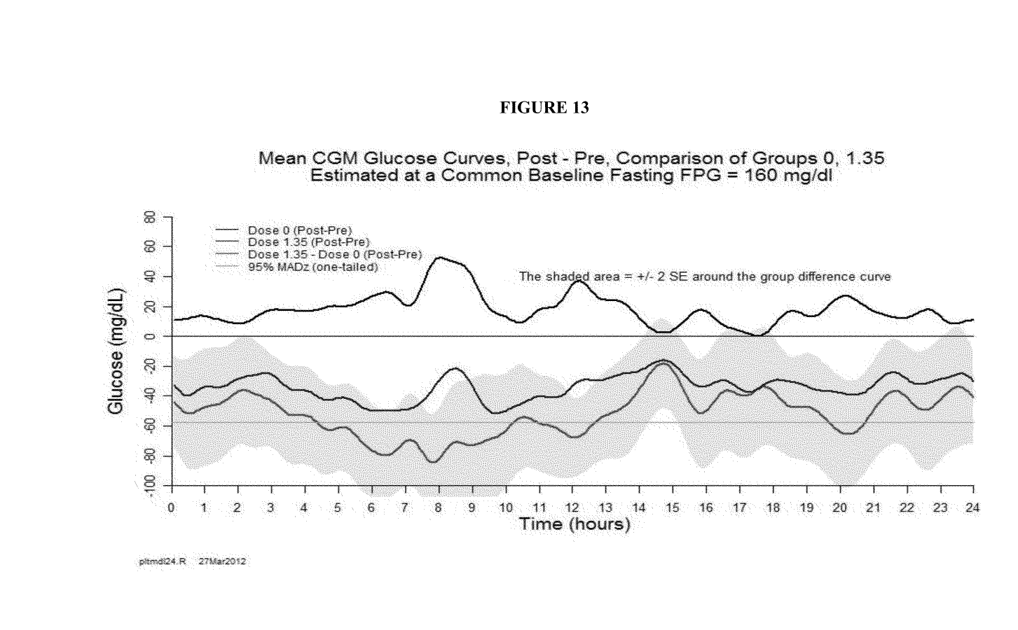

The pharmacodynamic analysis consisted of all subjects dosed who had sufficient data for PD analysis. Glymera™ displayed a clinically significant dose dependant effect on reduction in fasting plasma glucose (FIG. 10) and attenuation of glucose following a liquid meal challenge (FIG. 11) after 4 once weekly subcutaneous injections of Glymera™ at doses ranging from 0.6 to 1.35 mg/kg. Additionally, following multiple doses ranging from 0.6 to 1.35 mg/kg, Glymera™ was able to significantly reduce mean average daily glucose over the 7 day period following the fourth dose of study drug as measured by continuous glucose monitoring (CGM) (FIG. 12) which translates into a clinically meaningful reduction in imputed A1c of between −1.0% to -1.8% (placebo adjusted). Glymera™'s effects appear to be more pronounced during the morning hours as displayed in FIG. 13.

Currently marketed daily or weekly GLP-1 receptor agonists when given alone (monotherapy) report A1c reductions in the range of 0.7% to 1.5% following 26 to 52 weeks of treatment, whereas Glymera™ was capable of achieving an imputed reduction in A1c as high as 1.9% following 4 weeks of dosing.

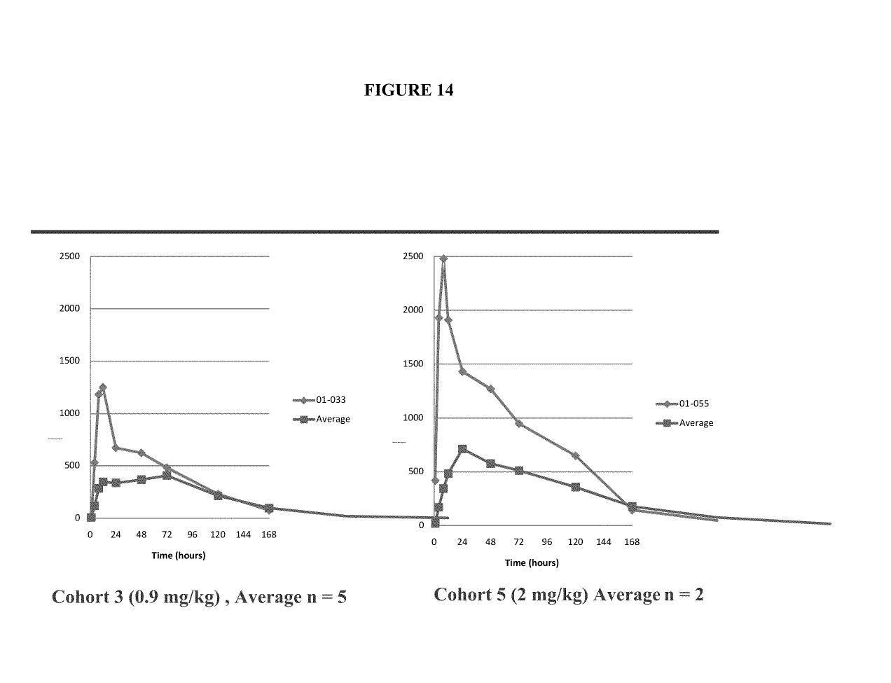

With respect to safety, Glymera™ displayed a highly favorable tolerability profile as compared to other currently marketed GLP-1 receptor agonists. Two subjects that experienced nausea, exhibited a significantly different PK profile, as shown in FIG. 14. Specifically, these subjects experienced a rapid exposure in Glymera™ levels of >1000 ng/ml, which is 2 to 3 fold greater than the amount necessary to elicit a significant PD effect (EC50 of 373±185 ng/mL as measured by glucose response following MMTT). The PK profile shown in the left panel was based on 0.9 mg/kg Glymera™. Mild nausea started about 6 hours post-dose and reportedly resolved at around 43 hours post-dose. The PK profile shown in the right panel was based on 2 mg/kg Glymera™. Mild nausea reportedly occurred at about 2 hours post dose with a duration of 4 days, and with moderate vomiting at about 4 hours post dose. Without being bound by theory, it is believed that this PK profile triggers nausea, and which is avoided with the sustained release profile experienced by most participants.

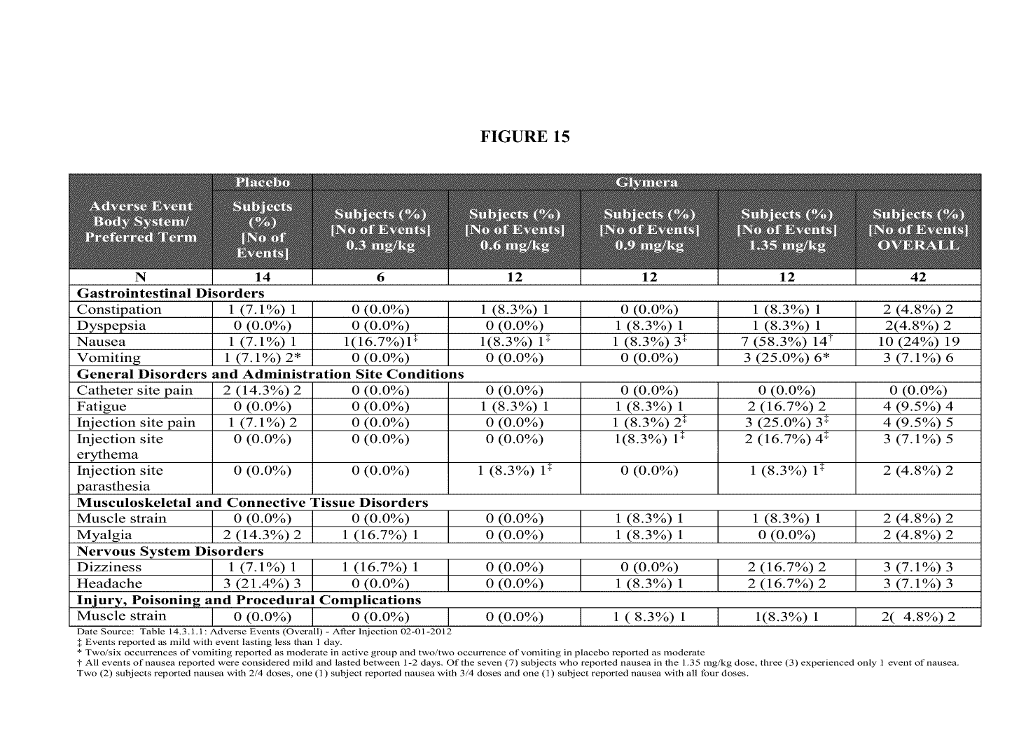

No SAEs or dose limiting toxicities were reported (FIG. 15). All adverse events were mild or moderate in severity. There were no dose related abnormal trends in change in laboratory parameters (Chem-12, CBC, LFTs, amylase/lipase, Lipids or Calcitonin), vital signs or ECGs that would indicate a safety concern. The most common reported adverse events (whether or not considered related to study drug) are listed below. One subject reported symptoms of hypoglycemia (plasma glucose 71 mg/dl) at about 6 hours after dosing which was self treated with oral carbohydrates.

Of the 42 subjects who received more than one dose of active study drug, one subject developed a low level non-neutralizing antibody response to Glymera™ with no associated injection site reactions. One subject developed a low level non-neutralizing antibody response to native GLP-1. This subject reported mild injection site erythema following the second dose that did not proliferate with subsequent injections.

Following 4 weeks of once weekly SC doses ranging from 0.6 to 1.35 mg/kg, Glymera™ displayed a clinically significant dose dependent effect on reduction in fasting plasma glucose and was able to significantly reduce mean average daily glucose over the 7 day dosing interval with minimal loss of glycemic control. This translates into a clinically meaningful reduction in imputed A1c of between −1.0% and −1.8% (placebo adjusted). Furthermore Glymera™ was capable of attenuating the rise in glucose (AUC_{0-240 minutes}) following a liquid meal challenge.

The rate-controlled exposure as a consequence of slow absorption from the site of injection, consistent with the ability of the ELP technology to control drug release at the site of injection, may enhance the overall gastrointestinal tolerability with maximal efficacy. There was minimal accumulation with repeated administration consistent with the half-life and once weekly dosing frequency.

Glymera™ was generally well tolerated with no clinically relevant safety signals that would preclude further development as a once weekly treatment for hyperglycemia in patients with type 2 diabetes.

### Example 3

### Modeling of Nausea Rate as a Function of Pk Parameters

Logistic model is used to model the Nausea rate Pₙₐᵤ(or the probability of nausea events) given the concentration Cₚₖ and rate of change Rₚₖ of PK.

Log it(Pₙₐᵤ)=α+β₁*Cₚₖ+β₂*Rₚₖ (1)

where α, β1 and β2 are the model coefficients. Since PK is estimated only at specific time, i.e., 1 hour, 4 hour, 8 hour, etc, the corresponding PK concentration Cₚₖ and rate of change Rₚₖ at the nausea onset time is estimated using Spline. The whole analysis was done in R and SAS. Repeated measure effects are not considered.

The following procedures were used to fit the logistic model:
1. Find all Nausea events whose onset time are within 7 days of Dose 1 and Dose 4.
2. Create a dataset including all subjects. Two variables—Nausea and onset are created. If a subject does have Nausea at that time point, then Nausea=1, otherwise Nausea=0.
3. Use R to fit Spline for each subjects' PK profile
4. For each subject, estimate the PK concentration Cₚₖ and rate of change Rₚₖ at the time points obtained in step 1.
5. Fit the logistic model (1) from this dataset using SAS.

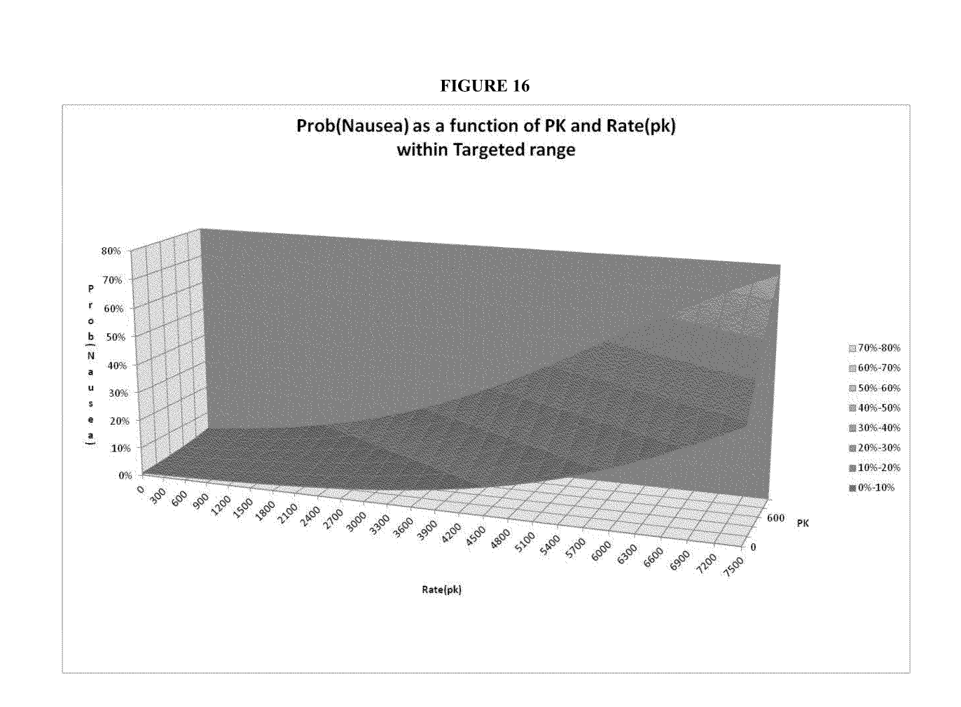

As shown in FIG. 16, at up to 1000 ng/ml (˜ED86), the nausea risk depends very little on absolute concentration, but is sensitive to the speed of increasing plasma drug concentration.

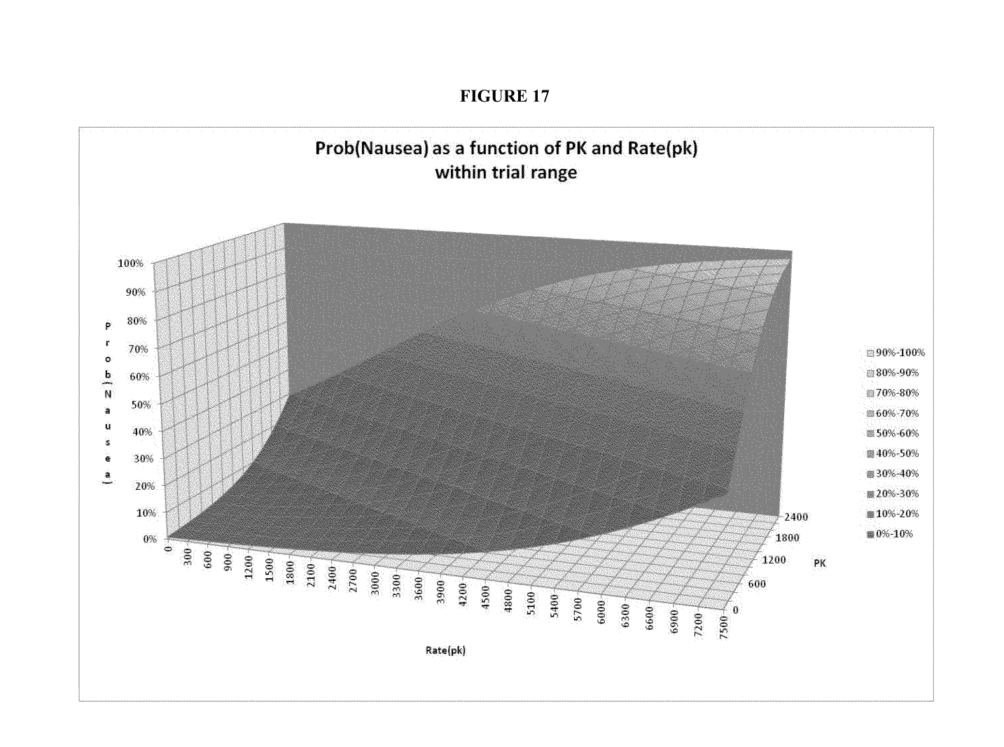

As shown in FIG. 17, within the observed range of absolute concentrations, the nausea risk increases considerably at the doses above those needed for glucose lowering effect.

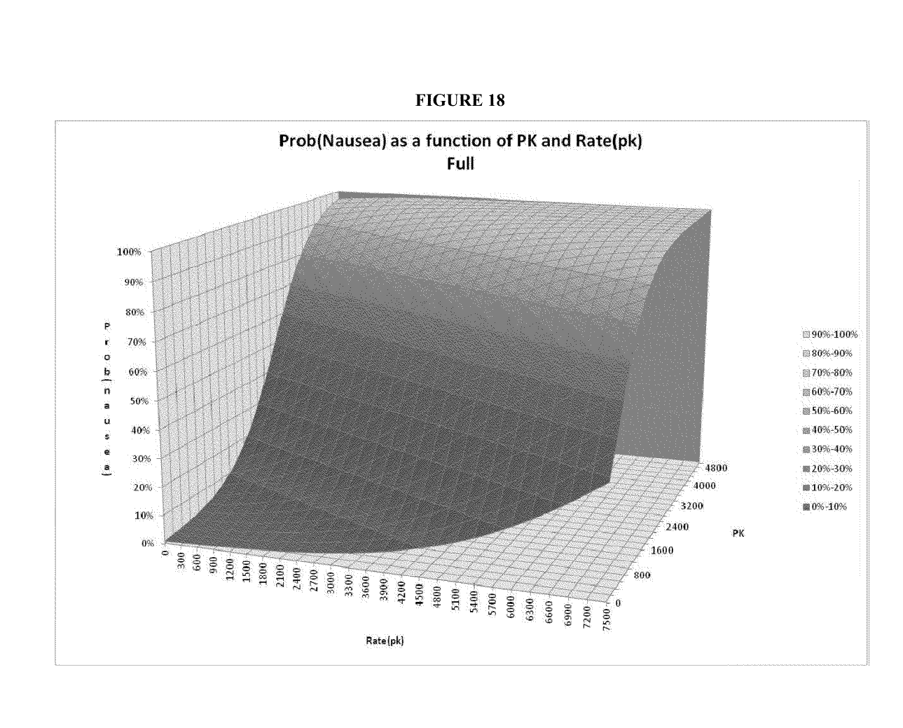

As shown in FIG. 18, including extrapolated data in the full model, very high absolute concentration of the GLP-1 fusion makes nausea almost a certainty, overwhelming the speed of increase as a precipitating factor.

All patents and non-patent publications cited herein are hereby incorporated by reference for all purposes.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. -109. (canceled)

110. A method of inducing weight loss in a patient in need thereof, comprising administering at least one effective dose of a GLP-1 receptor agonist, wherein the effective dose does not induce substantial appetite suppression in the patient.

111. The method of claim 110, wherein the effective dose or regimen does not induce substantial nausea in the patient.

112. The method of claim 110, wherein the effective dose or regimen does not require a substantial reduction in food intake in the patient to control weight gain, induce weight loss, or prevent weight gain.

113. The method of claim 110, wherein the patient is overweight or obese.

114. The method of claim 110, wherein the patient has metabolic disease.

115. The method of claim 110, wherein the patient is diabetic or pre-diabetic.

116. The method of claim 110, wherein the GLP-1 receptor agonist is GLP-1 7-36, GLP-1 7-37, exendin, or a derivative or homolog thereof

117. The method of claim 116, wherein the GLP-1 receptor agonist is formulated for sustained release, and/or exhibits slow uptake to the patient's circulation when administered.

118. The method of claim 117, wherein the slow uptake is effected by fusion of the GLP-1 receptor agonist to a second protein.

119. The method of claim 118, wherein the second protein forms a reversible hydrogen-bonded matrix at the body temperature of the subject.

120. The method of claim 119, wherein the second protein is an elastin-like protein (ELP).

121. The method of claim 120, wherein the amino acid sequence of the ELP is:
VPGXG, where each X is selected from V, G, or A; AVGVP; IPGVG; or LPGVG.

122. A method of preventing weight gain in a patient in need thereof, comprising administering at least one effective dose of a GLP-1 receptor agonist, wherein the effective dose does not induce substantial appetite suppression in the patient.

123. A method of controlling weight in a patient in need thereof, comprising:
administering a GLP-1 receptor agonist regimen to the patient, the regimen being effective to maintain therapeutic serum levels of said GLP-1 receptor agonist below a nausea-inducing threshold level, and/or avoid a nausea-inducing rise in GLP-1 levels.

124. The method of claim 123, wherein the effective dose or regimen does not induce substantial nausea in the patient.

125. The method of claim 123, wherein the effective dose or regimen does not require a substantial reduction in food intake in the patient to control weight gain, induce weight loss, or prevent weight gain.

126. The method of claim 123, wherein the patient is overweight or obese.

127. The method of claim 123, wherein the patient has metabolic disease.

128. The method of claim 123, wherein the patient is diabetic or pre-diabetic.

129. The method of claim 123, wherein the GLP-1 receptor agonist is GLP-1 7-36, GLP-1 7-37, exendin, or a derivative or homolog thereof

130. The method of claim 123, wherein the GLP-1 receptor agonist is formulated for sustained release, and/or exhibits slow uptake to the patient's circulation when administered.

131. The method of claim 130, wherein the slow uptake is effected by fusion of the GLP-1 receptor agonist to a second protein.

132. The method of claim 131, wherein the second protein forms a reversible hydrogen-bonded matrix at the body temperature of the subject.

133. The method of claim 132, wherein the second protein is an elastin-like protein (ELP).

134. The method of claim 133, wherein the amino acid sequence of the ELP is:
VPGXG, where each X is selected from V, G, or A; AVGVP; IPGVG; or LPGVG.


SEQUENCE LISTING (21 sequences)

SEQ ID NO: 1 - protein, 4 aa, Artificial Sequence
  ELP tetrapeptide
VPGG

SEQ ID NO: 2 - protein, 4 aa, Artificial Sequence
  ELP tetrapeptide
IPGG

SEQ ID NO: 3 - protein, 5 aa, Artificial Sequence
  ELP pentapeptide
VPGXG

SEQ ID NO: 4 - protein, 5 aa, Artificial Sequence
  ELP pentapeptide
AVGVP

SEQ ID NO: 5 - protein, 5 aa, Artificial Sequence
  ELP pentapeptide
IPGXG

SEQ ID NO: 6 - protein, 5 aa, Artificial Sequence
  ELP pentapeptide
IPGVG

SEQ ID NO: 7 - protein, 5 aa, Artificial Sequence
  ELP pentapeptide
LPGXG

SEQ ID NO: 8 - protein, 5 aa, Artificial Sequence
  ELP pentapeptide
LPGVG

SEQ ID NO: 9 - protein, 6 aa, Artificial Sequence
  ELP hexapeptide
VAPGVG

SEQ ID NO: 10 - protein, 8 aa, Artificial Sequence
  ELP octapeptide
GVGVPGVG

SEQ ID NO: 11 - protein, 9 aa, Artificial Sequence
  ELP nonapeptide
VPGFGVGAG

SEQ ID NO: 12 - protein, 9 aa, Artificial Sequence
  ELP nonapeptide
VPGVGVPGG

SEQ ID NO: 13 - protein, 636 aa, Artificial Sequence
  GLP-1 receptor agonist
HGEGTFTSDVSSYLEGQAAKEFIAWLVKGRGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 14 - protein, 30 aa, Homo sapiens
HAEGTFTSDVSSYLEGQAAKEFIAWLVKGR

SEQ ID NO: 15 - protein, 30 aa, Homo sapiens
HGEGTFTSDVSSYLEGQAAKEFIAWLVKGR

SEQ ID NO: 16 - protein, 38 aa, Homo sapiens
HGEGTFTSDLSKQMEEEAVRLFEWLKNGGPSSGAPPPS

SEQ ID NO: 17 - protein, 37 aa, Homo sapiens
HSQGTFTSDYSKYLDSRRAQDFVQWLMNTKRNKNNIA

SEQ ID NO: 18 - protein, 37 aa, Homo sapiens
HSEGTFTSDYSKYLDSRRAQDFVQWLMNTKRNRNNIA

SEQ ID NO: 19 - protein, 29 aa, Homo sapiens
HSQGTFTSDYSKYLDSRRAQDFVQWLMNT

SEQ ID NO: 20 - protein, 33 aa, Homo sapiens
HADGSFSDEMNTILDNLAARDFINWLIQTKITD

SEQ ID NO: 21 - protein, 29 aa, Homo sapiens
YAEGTFISDYSIAMDKIRQQDFVNWLLAQ